(19) United States
(12) Patent Application Publication
(10) Pub. No.: US 2003/0003069 A1
(43) Pub. Date: Jan. 2, 2003

(54) Multiple phase foaming personal cleansing products

(76) Inventors: John C. Carson, Union City, NJ (US); Albert Zofchak, Matawan, NJ (US)

Correspondence Address: Henry D. Coleman, COLEMAN SUDOL, LLP, 14th Floor, 708 Third Avenue, New York, NY, 10017-4101, US

(21) Appl. No.: 09/826,482

(22) Filed: Apr. 4, 2001

(51) Int. Cl. A61K 7/06

(52) U.S. Cl. 424/70.1

(57) ABSTRACT

The present invention relates to personal care foaming and cleansing products comprising multiple phases which are separate and distinct and comprise at least one surfactant solution having a density or specific gravity of approximately 1.0. In a two-phase composition, a second separated phase may comprise cosmetically acceptable oily materials as emollients/conditioners having a density greater than that of the surfactant solution. In a three phase composition, an additional phase may comprise a low density emollient/conditioning compound(s) as an upper third phase. Thus, two, three or even four phase foaming bath products, shampoos and skin cleansers can be produced that can be differentially colored to enhance the visual separation of the layers and will provide foaming, cleaning, conditioning and emolliency in a unique and attractive manner.

### FIELD OF THE INVENTION

The present invention relates to personal care products such as cosmetics and toiletries. More specifically, the present invention relates to foaming skin and hair cleansing products such as bubble baths, skin cleansers and shampoos.

### BACKGROUND OF THE INVENTION

Foaming bubble bath products are typically composed of water, foaming and cleansing surfactants, foam stabilizers, skin conditioners, opacifiers and pearling agents (if desired), viscosity control agents, preservatives, colors and fragrance ingredients. The specific components are chosen to meet the desired product performance criteria. That is, if the product is to be clear, then the total assembly of the components is chosen to be compatible and produce a stable, clear solution. If, an opaque or pearly product is desired, then the components (without the opacifier/pearling agent) need not produce a clear solution, but the total product must be stable and, for a conventional product, can not separate.

In the past, there have been two phase bubble bath products based upon mineral oil that would separate to the top of a solution composed of water, a surfactant (such as sodium laureth sulfate or ammonium laureth sulfate), cocamide DEA or cocamide MEA and ethanol. The ethanol is a critical ingredient for several reasons. First, the ethanol helps to break the emulsion formed by the surfactant/water solution and the mineral oil. Second, since ethanol and mineral oil are mutually insoluble this enhances the separation of the mineral oil phase from the surfactant solution. Third, the ethanol reduces the viscosity of surfactant solution allowing more rapid separation of the phases (Stokes Law). It is well recognized in the art, that other materials such as glycols, polyhydroxy alcohols, many salts, hydrotropes and some viscosity control agents (such as xylene sulfonate salts) will produce results similar to those seen with ethanol.

In addition to the product described above, there are two and three phase bath oil products which currently exist commercially. These are products which separate into distinct layers and can be differentially colored to produce very appealing visual effects. These products do not contain surfactants, do not produce a foam or lather in use, do not provide cleansing and are intended for use as emollient bath oils and fragrance vehicles.

To date, the only multiple phase skin cleansing products available are those which contain solid particulates such as ground nut shells, jojoba wax beads, polyethylene beads, etc. These materials provide a novel visual effect for the product as well as producing a “scrubbing” or “skin exfoliation” effect during use.

Two phase shampoos (such as “Twice as Nice Shampoo”) have also been commercially produced. This product contained mineral oil as the upper (conditioner) phase and a low viscosity, surfactant blend as the lower phase. The blend was similar to that used for the bubble bath mentioned previously in that it contained an ethoxylated lauryl sulfate surfactant, an amide foam stabilizer and it used ethanol to control viscosity and break the emulsion that forms with the mineral oil phase when the product is shaken. To use, the bottle was shaken to temporarily mix the product, the product was then dispensed, applied to wet hair, which was shampooed, rinsed and styled in the usual manner. Thus, the product was able to provide foaming, cleansing and hair conditioning from a single usage. Subsequently, upon standing, the product would again separate into two layers.

### OBJECTS OF THE INVENTION

It is an object of the invention to provide novel two phase foaming and cleaning surfactant systems in which both phases are liquid and the “conditioning” or oily phase is on the bottom.

It is an additional object of the invention to provide three phase foaming and cleaning surfactant systems.

It is a further object of the invention to provide a three phase foaming and cleaning surfactant system wherein all three layers are liquids.

It is an object of this invention, but not a necessary condition, to provide novel three phase foaming and cleaning surfactant systems in which all phases are liquid and clear.

It is yet another object of the invention to provide four phase foaming and cleaning surfactant systems.

It is still another object of the invention to provide a four phase foaming and cleaning surfactant system in which there is at least one phase which is a substantially solid phase which is added to compositions according to the present invention to assist in the exfoliation of the skin and to provide a “scrubbing” action and feel on the skin.

Any one of these and/or other objects of the present invention may be readily gleaned from a review of the description of the invention which follows.

### SUMMARY OF THE INVENTION

The present invention relates to compositions comprising at least two distinct phases. In a two phase aspect of the present invention, a two phase composition comprises an upper layer and a lower layer, the upper layer comprising a surfactant solution capable of foaming upon application of said composition to a surface and having a specific gravity of less than about 1.0, preferably between about 0.85 and about 1.0, even more preferably about 0.95 to about 1.0, said lower layer comprising at least one non-defoaming high density ester, preferably an aromatic ester, having a specific gravity greater than about 1.0 (preferably, between about 1.0 and about 1.20 and more preferably about 1.0 to about 1.1), said lower layer being a hydrophobic layer exhibiting emollient and/or conditioning characteristics comprising at least one high density ester, preferably an aromatic ester, said high density ester having a minimum tendency to defoam said surfactant solution.

In an alternative embodiment according to the present invention, a three phase composition is contemplated comprising three distinct layers: a lower non-defoaming emollient or conditioning layer, a middle layer comprising a surfactant solution capable of foaming upon application of said composition to a surface and an upper layer comprising an emollient or conditioner, said lower layer comprising at least one high density ester, preferably an aromatic ester, having a specific gravity of greater than about 1.0 (preferably, between about 1.0 and about 1.20 and more preferably about 1.0 to about 1.1), said high density ester having a minimum tendency to defoam said surfactant solution, a middle layer comprising a surfactant solution capable of foaming upon application of said composition to a surface, and having a specific gravity ranging from about 0.95 to about 1.05, preferably about 1.00; and a third or upper layer comprising an oily layer having emollient characteristics comprising a non-defoaming emollient or conditioner preferably selected from the group consisting of a C₁₀ to C₃₀ aliphatic hydrocarbon, mineral oil, a fatty acid ester or a polysiloxane emollient or conditioner such as a polydimethylsiloxane or a cyclomethicone, said third or upper layer having a specific gravity ranging from about 0.75 to about 0.95, preferably about 0.8 to about 0.95, more preferably about 0.85 to about 0.95. In preferred aspects according to this aspect of the present invention, the first or lower layer has a specific gravity which is at least about 0.05 greater than said second or middle layer and said second or middle layer has a specific gravity which is at least about 0.05 greater than than the third or top layer.

In a separate embodiment, a four phase composition for application to the skin is contemplated, said composition comprising a lower or first non-defoaming emollient or conditioning layer, a second layer comprising a surfactant solution capable of foaming upon application of said composition to a surface, an upper or third layer comprising an emollient or conditioner and a fourth layer comprising a solid exfoliation agent to assist in exfoliating the skin and to provide a “scrubbing” action and feel on the skin, the lower layer comprising at least one high density ester, preferably an aromatic ester having a specific gravity of greater than about 1.0 (preferably, between about 1.0 and about 1.20, more preferably about 1.0 to about 1.1), said high density ester having a minimum tendency to defoam said surfactant solution, the middle layer comprising a surfactant solution capable of foaming upon application of said composition to a surface such as the skin, said middle or second layer having a specific gravity ranging from about 0.95 to about 1.05, preferably about 1.00; and a third or upper layer comprising an oily layer having emollient characteristics comprising a non-defoaming emollient or conditioner, preferably a C₁₀ to C₃₀ aliphatic hydrocarbon, mineral oil, a fatty acid ester or a polysiloxane emollient or conditioner such as a polydimethylsiloxane or a cyclomethicone, said third or upper layer having a specific gravity ranging from about 0.75 to about 0.95, preferably about 0.8 to about 0.95, more preferably about 0.85 to about 0.95. In preferred aspects according to this aspect of the present invention, the first or lower layer has a specific gravity which is at least about 0.05 greater than said second or middle layer and said second or middle layer has a specific gravity which is at least about 0.05 greater than than the third or top layer. In this four-phase aspect of the present invention, when the fourth layer is a solid phase (the preferred aspect), the fourth layer, in a settled state, will appear in the composition at the lowest layer in the composition and upon which the other three layers will rest. In other aspects according to the invention, the fourth layer may comprise an exfoliating agent or mixture of agents as a liquid or preferably a solid, wherein the fourth layer may be found in the composition anywhere from the lowest layer to the upper layer depending upon the specific gravity of the exfoliating layer relative to the other layers in the composition.

In preferred aspects according to the present invention, each of the layers is a liquid layer and the viscosity of each layer is less than about 1000 cps. Viscosities of the different layers may range from below 1.00 cps to as high as 1,000 cps or more, with a preferred range of about 1 cps to about 500 cps, more preferably about 1 cps to about 25-50 cps. In preferred aspects according to the present invention which relate to compositions which rapidly separate into layers, preferred viscosities are those which are lower (i.e., closer to 1.00 cps) in order to promote a rapid separation, although the viscosities may be adjusted to reflect a desired rate of mixing of the layers upon shaking and separation of the layers after shaking. As a guide, those layers having a higher viscosity will tend to separate more slowly and those layers having a lower viscosity (i.e., closer to 1.0) will separate more rapidly, although the size of a layer relative to other layers in the composition, as well as the difference in specific gravities of the different layers may also markedly influence the rate of separation.

It has been surprisingly discovered that high density esters (defined as esters having a specific gravity greater than 1.0) and more specifically, high density esters of aromatic acids, have a minimal tendency to defoam surfactant solutions. It is well known that many different types of esters including, but not limited to, diisopropyl adipate, isopropyl palmitate, dioctyl adipate and octyl palmitate reduce both the volume and stability of the foam which is produced by surfactants. It has been found that aromatic esters (“high density aromatic esters”) such as, for example, propylene glycol benzoate, dipropylene glycol benzoate, dipropylene glycol dibenzoate, propylene glycol dibenzoate, octyl methoxycinnamate, menthyl anthranilate, octyl salicylate, octyl cinnamate, and octocrylene, among others, do not reduce either the volume or stability of foams produced by surfactants. This is an unexpected result.

This finding, coupled with the fact that these aromatic acid esters tend to have a specific gravity greater than 1.0, has led to the development of two phase foaming surfactant systems comprising an oily, “conditioning” layer as the lower layer and a foaming, cleansing surfactant layer on top. Pursuant to the present invention, the layers can be differentially colored providing a visually appealing as well as functionally effective foaming, cleansing and “conditioning” system.

In another aspect of the present invention, a novel three phase foaming and cleaning surfactant composition comprises three distinct layers wherein there is a top “oily” layer having emollient characteristics comprising low density (specific gravity less than 1.0) aliphatic hydrocarbons, fatty acid esters, dimethylsiloxanes or cyclomethicones that do not appreciably defoam surfactant solutions, a middle layer containing foaming and cleaning surfactants and a lower layer comprising high density esters (having a specific gravity greater than 1.0) which are not miscible with the low density compounds of the top layer or the middle surfactant layer.

It is a final object of this invention to provide novel four phase foaming and cleaning surfactant systems in which the three phases are as described previously and the fourth phase is an added solid phase, such as, but not limited to: ground plant materials, finely divided polyethylene, hydrogenated jojoba oil spheres, sand or pumice. These materials are added to assist in the exfoliation of the skin and to provide a “scrubbing” action and feel on skin.

### DETAILED DESCRIPTION OF THE INVENTION

The following terms shall be used throughout the specification to describe the present invention.

The term “high density aromatic ester” is used throughout the specification to describe an emollient and/or conditioning ester which contains an aromatic group such as a substituted or unsubstituted phenyl, naphthyl, anthracene, phenanthrene or related aromatic group, has emollient and/or conditioning characteristics and a specific gravity which is greater than 1.0. Exemplary high density aromatic esters for use in the present invention include propylene glycol benzoate, dipropylene glycol benzoate, propylene glycol dibenzoate, dipropylene glycol dibenzoate, octyl methoxycinnamate, menthyl anthranilate, octyl salicylate and octocrylene, among numerous others.

The term “surfactant” is used to describe compositions according to the present invention which are included in the surfactant solution phase in compositions according to the present invention for their ability to solubilize and remove oils and other materials from a surface exposed to the surfactant solution. Preferred surfactants for use in the present invention are those surfactants which produce foams upon exposure to a surface. Exemplary surfactants for use in the present invention include nonionic, anionic, cationic, amphoteric and zwitterionic surfactants, with anionic surfactants being preferred. Preferred anionic surfactants for use in the present invention include, for example, alkyl sulfates, alkylether sulfates, alkyl benzene sulfonates, alpha olefin sulfonates, N-alkyl sarcosinates, alkyl sulfosuccinates, alkyl phosphates, alkylether phosphates and alkyl or alkylether carboxylic acid salts, among others.

The term “non-defoaming” is used to describe emollients and/or conditioners which are used in compositions according to the present invention. By non-defoaming it is meant that the emollient or conditioner will not appreciably reduce the rate of foaming or the foam volume which occurs when the surfactant is rubbed while in contact with a surface or as evaluated in a standardized foam test method which is well known to those of ordinary skill in the art.

The term “separation enhancer” is used throughout the specification to describe an additive or compound which is added to compositions according to the present invention which enhances the tendency of the phases in compositions according to the present invention to separate after mixing. Separation enhancers for use in the present invention include, for example, salts such as sodium chloride, ammonium chloride, acetamidopropyltrimonium chloride, tetrasodium ethylenediaminetetraacetate, trisodium phosphate, hydrotropes, such as sodium and ammonium xylene sulfonates, sodium alkyl disulfonates, solvents, particularly alcoholic solvents such as ethanol, isopropanol, ethoxy diglycol, glycols and polyhydroxy compounds such as propylene glycol, methyl propane diol, butylene glycol, hexylene glycol, glycerin, dextrose, sorbitol, sucrose, fructose and other sugars. Separation enhancers for use in the present invention should be soluble only in the surfactant phase and should not be miscible with or soluble in the separating phases. Separation enhancers are used in surfactant phases according to the present invention in effective amounts for enhancing the separation of phases within the present compositions. Preferably, a separation enhancer may be used in compositions according to the present invention in effective amounts ranging from about 0.5% to about 25% or more by weight, more preferably about 5% to about 15% by weight.

The term “phase” means a distinct layer which appears in compositions according to the present invention after a sufficient settling period (preferably, at least about 1 minute, more often about 5 minutes or more up to about 30 minutes, and in certain embodiments, up to several days or more). Compositions according to the present invention comprise two, three or four distinct phases or layers.

The term “settling” or settled shall be used to describe compositions which have had sufficient time to settle out and to separate into distinct phases.

The term “effective” is used to describe an amount of a particular component which is used in or added to the compositions according to the present invention to provide an intended effect. For example, an effective amount of an emollient is that amount effective to provide emolliency to a final composition. Likewise, an effective amount of a surfactant or conditioner is that amount effective to produce the intended effect of adding the particular component to the compositions of the present invention.

The term “hydrotrope” is used throughout the specification to describe a compound that is soluble in the water phase of the formulation and that functions to solubilize the surfactants in the water phase. Hydrotropes for use in the present invention include, for example, sodium and ammonium xylene sulfonates, sodium alkyl disulfonates, solvents, particularly alcoholic solvents such as ethanol, isopropanol, ethoxy diglycol, glycols and polyhydroxy compounds such as propylene glycol, methyl propane diol, butylene glycol, hexylene glycol, glycerin, dextrose, sorbitol, sucrose, fructose and other sugars

The present invention is, in essence, a surfactant solution intended for personal cleansing, having the added benefit of the emolliency/conditioning characterisitics of the additional, separate phases. As such, it must be formulated of high foaming, mild detergents at sufficient percentages in the product to effect cosmetically acceptable cleansing and foaming. Such effective percentages are, at the same time, those that readily cause the emulsification of oily materials—which is one of the primary functions of a detergent. Therefore, providing a detergent solution with emollient (oily) character in an at least a two phase system is desirable, but causing an oily phase to separate from a surfactant solution of sufficient strength to clean is, on the face of it, an inherently difficult task.

There are, however, several effective general approaches to producing preferred compositions according to the present invention which facilitate the separation of phases as clear liquids. These include, but are not limited to the following:

1. Choosing chemical components to be formulated within individual the layers that have the greatest differences in densities. By utilizing components in a given layer of a composition according to the present invention that have maximally different densities from components in other layers, this will provide compositions according to the present invention which will more readily separate. According to Stokes Law, the greater the difference in density between two compositions, the faster will be the separation rate of the two compositions.

2. Keeping all phase viscosities to a minimum. This will allow layers of different densities to separate quickly. The greater the viscosity of a layer, the slower the layer will be to separate from another layer.

3. Choosing surfactants which are more water soluble (i.e., that have a higher C.M.C. or higher HLB) because they tend to be poorer emulsifiers, as well as using the minimum amount of surfactant needed to achieve the desired performance of the composition.

4. Adding optional ingredients which, in certain embodiments, will assist the separation of the layers (“separation enhancers”), such as salts including, for example, sodium chloride, ammonium chloride, acetamidopropyltrimonium chloride, Na₄EDTA, Na₃PO₄, hydrotropes, including, for example, ammonium and sodium xylene sulfonates, potassium xylene sulfonates, sodium alkyl disulfonates and potassium alkyl disulfonates, solvents, for example, alcohols such as ethanol, isopropanol, ethoxy diglycol, glycols and amides such as acetamide, MEA and lactamide MEA, as well as polyols (i.e., polyhydroxy compounds) such as propylene glycol, methylpropane diol, butylene glycol, hexylene glycol, glycerin, dextrose, sorbitol and sucrose. The separation enhancer to be used in the present invention should be soluble only in the surfactant phase and not miscible or soluble in the separating phases or other oily/conditioning phases. The amount of the separation enhancer included in compositions according to the present invention is an effective amount to improve the separation of the layers of the composition. This amount will range from about 0.5% by weight of the final composition to as much as 25% by weight of the final composition, with a preferred range being about 5.0% to about 15% by weight of the final composition.

The use of some or all of these methods to improve separation can result in formulations that will separate as desired.

A further aspect of the present invention resides in the fact that many cosmetic esters, including many vegetable oils, will interfere with the amount of foam that a surfactant will produce by reducing the volume of foam generated and by reducing the foam's stability. Thus, in the present invention it is generally necessary to chose ingredients such as mineral oil and other aliphatic hydrocarbons which will separate from the surfactant solution and will not defoam the surfactant during use.

In the pursuit of the present invention, Applicants have surprisingly discovered several high density aromatic esters which have densities which are greater than 1.0 and which do not defoam or at least appreciably minimize defoaming of surfactant solutions. These aromatic esters include, for example, dipropylene glycol benzoate, dipropylene glycol dibenzoate, propylene glycol benzoate, propylene glycol dibenzoate, salicylate esters, for example, 2-ethylhexyl salicylate, menthyl anthranilate, octocrylene and cinnamate esters such as 2-ethylhexyl methoxycinnamate, among numerous others.

In addition to high density aromatic ester emollients/conditioners, compositions according to the present invention may include low density emollients and/or conditioners (these compounds have densities or specific gravities less than 1.0, preferably from about 0.8 to about 0.95, more preferably about 0.85 to about 0.95) including for example, long chain aliphatic compounds such mineral oil, squalane, squalene, difatty esters such as Jojoba oil, oleyl oleate and oleyl erucate, among others, as well as polydimethylsiloxanes (especially, short chain, low molecular weight), cyclomethicones, such as decamethylcyclopentasiloxane. It has been surprisingly discovered that cyclomethicones (which are normally thought to defoam surfactant systems) can be used as all or part of the separating upper phase and, when used in combination with a surfactant having a high HLB or CMC, can be formulated to have little effect upon foaming while producing clear, three phase systems with good skin feel, good hair effects and good afterfeel on skin and hair.

The above-described low density emollients/conditioners are added to compositions according to the present invention as optional components in order to produce three (or four) layer compositions. Because the density or specific gravity of these compounds is less than the surfactant solution (which is approximately 1.0), the layer which comprises these compounds will float on the surfactant solution and generally become the upper layer of the present composition. These compositions will generally (but not necessarily) comprise either the third (upper) layer in a three layer composition or the fourth layer in a four layer composition.

In general, the amount of low density emollients/conditioners used in the upper layer according to the present invention ranges from about 0.5% to about 25% by weight of the composition, preferably about 1.0% to about 10% by weight of the final three (or four) layer composition.

The surfactant solution phase (middle or upper phase of compositions according to the present invention) generally comprises an aqueous solution or emulsion of a surfactant in water. The amount of surfactant included within the surfactant solution phase is an effective amount generally within the range from about 5% to about 30% by weight of the final composition, preferably about 10% to about 25% by weight of the final composition. The amount of surfactant within the surfactant solution phase generally falls within the range of about 5% to about 40% by weight, more preferably about 15% to about 30% by weight.

The surfactant to be chosen for inclusion in compositions according to the present invention includes an effective amount of a surfactant which will readily solubilize in water, will produce a foam when exposed to a surface and when combined with water, will have a density which falls within the range of about 0.95 to about 1.05, most preferably about 1.0. Preferred surfactants for use in the present invention, include, for example, alkyl sulfates, alkylether sulfates, alkyl benzene sulfonates, alpha olefin sulfonates, N-alkyl sarcosinates, alkyl sulfosuccinates, alkyl phosphates, alkylether phosphates and alkyl or alkylether carboxylic acid salts, among others.

In the surfactant solution phase, in addition to water and surfactant, this phase may optionally comprise at least one additional agent selected from the group consisting of foam boosters, foam stabilizers, polymers, solvents, humectants, hydrotropes, viscosity control agents, density (specific gravity) control agents, emulsion separation agents, colors, fragrances and preservatives. The surfactant solution phase provides the cleansing, foaming and detergency for the product, and it is maintained at a density or specific gravity within a range falling between those of the upper and lower phases.

The present invention allows one of ordinary skill to readily produce unique personal cleansing compositions that are composed of two, three or four separate phases which may be separately and individually colored to produce visually impactful products. The products according to the present invention are intended to be visually unique and it is an object of this invention to create the distinct impression of having multiple phases. Therefore, it is a preferred aspect of the present invention that all phases are clear upon separation. Also, it is a preferred aspect of the present invention that the phases separate rapidly. The more rapidly a shaken product separates into layers, and the clearer those layers are upon settling, the stronger will be the impression that it makes as a unique product. It is also a preferred aspect of the present invention that the individual layers be individually and uniquely colored to enhance the appearance of separation.

In general, in the present compositions, the volume of the separated phase, or phases, must be sufficiently large so as to be readily apparent in the package. Notwithstanding the desirability of providing distinct layers in compositions according to the present invention, the total oil phase can not be so large as to overwhelm or “swamp” the functioning of the product as a cleanser. Preferably, the surfactant layer of compositions according to the present invention comprises about 20% to about 97.5% by weight of the final composition, more preferably about 30% to about 90% by weight, even more preferably about 60% to about 85% (even more preferably at least about 50% within this range) by weight of the final composition. In two layer compositions according to the present invention, the surfactant layer comprises about 50% to about 95% by weight of the composition, with the oil phase comprising at least one high density aromatic ester in an amount ranging from about 2.5% to about 50% by weight (preferably, at least about 5% by weight within this range) of the two layer composition.

In three layer compositions according to the present invention, the third or upper phase in the present compositions comprises a low density emollient/conditioner as otherwise described in amounts ranging from about 0.5% to about 35% by weight of the final composition, more preferably about 5% to about 15% by weight of the final composition. Low density emollients/conditioners for use in the present invention are selected from a number of materials including, for example, long chain aliphatic compounds such as C₁₀. to C₂₄ aliphatic compounds, mineral oils, squalane and squalene, difatty esters for example, Jojoba oil, oleyl oleate and oleyl erucate, short chained polydimethylsiloxane and cyclomethicones, including decamethylcyclopentasiloxane, among others. The specific gravity of this upper phase preferably ranges from about 0.75 to about 0.95, preferably about 0.8 to about 0.95 and in certain instances, more preferably, 0.85 to about 0.95.

In a further aspect of the present invention, a four layer composition according to the present invention is contemplated wherein said fourth layer is an exfoliating layer comprising a layer of solid, non-soluble particles which comprise at least one agent selected from the group consisting of ground plant materials, finely divided polyethylene, hydrogenated jojoba oil spheres, diatomaceous earth, sand, and pumice, among numerous other exfoliating agents. Exfoliating agents for use in the present invention include physical abrasion type exfoliating agents, a number of which are presented above. In this aspect of the present invention, the exfoliating agent comprises about 0.1% to about 20% by weight of the composition, more preferably about 0.5% to about 10% by weight of said composition. In general, this fourth layer, because of the density of the particles, tends to be located either at the bottom of the container or in association with one of the liquid layers.

Also, in a three phase product, there can be appreciable mutual solubility of the upper and lower oil phase materials, even though every effort is made to minimize their mutual solubility. This affects the volumes of the two phases that separate to the top and bottom. Therefore, achieving practical and aesthetically pleasing separated phase volumes in a three phase product is an effort of experimentation.

Compounding the above problem is also the solubility (or emulsifiability) of the oil phase (or phases) in the surfactant solution and the solubility of the surfactant solution in the oil phase or phases. This, of course, affects the clarity of the phases and the speed at which they separate. As more of the oil phase is emulsified into the water phase, the more opaque it becomes. Similarly, the oil phase or phases become more opaque as more of the surfactant phase is incorporated. A separation of the product into several opaque phases may be desirable and aesthetically pleasing and indeed, several example formulations of such products will be provided. But, achieving this effect is also a matter of experimentation.

In addition to affecting the clarity of the separated phases, the partial solubility of one phase in another phase, will also affect the phase volume. Thus, the amounts of each phase have to be adjusted experimentally in order to establish aesthetically pleasing phase volume ratios.

Finally, temperature dramatically affects the mutual solubilities of the phases. Thus, it has an effect on the phase volume ratios and can affect the clarity of the phases. Therefore, it becomes critical to evaluate the degree and quality of phase separation at a standardized temperature. As always, the products must be tested for physical stability at various temperatures, but, evaluation of phase separation should be carried out at the standardized temperature in addition to the test temperature.

The following non-limiting examples are provided as illustrations of the present invention.

EXAMPLE 1

| Ingredients | % |
|---|---|
| Sodium Lauroyl Sarcosinate (30% active) | 20.0 |
| Cocamidopropyl Betaine | 20.0 |
| Dipropyleneglycol Dibenzoate | 7.5 |
| Mineral Oil (70 ssu) | 7.5 |
| Deionized Water | 45.0 |
| Fragrance, Color, Preservative | QS |
| | 100.0 |

The separation of phases for this formula is quite good, although the upper layer is approximately twice as large as the bottom layer. This is an example of a formula in which the upper and lower layers are opaque and the middle layer is clear.

EXAMPLES 2 & 3

| | EXAMPLE 2 | EXAMPLE 3 |
|---|---|---|
| Ingredients | % | % |
| Disodium Laureth Sulfosuccinate | 20.0 | 30.0 |
| Cocamidopropyl Betaine | 20.0 | 30.0 |
| Mineral Oil (70 ssu) | 5.0 | 5.0 |
| Dipropyleneglycol Dibenzoate | 10.0 | 10.0 |
| Propylene Glycol | — | 10.0 |
| Deionized Water | 45.0 | 15.0 |
| Fragrance, Color, Preservative | QS | QS |
| | 100.0 | 100.0 |

The separation of phases in Example 2 is quite good with the upper phase representing about 7.5% of the product volume, the middle about 80% and the lower phases about 12.5% of the product volume. In this example, the top phase is opaque, the middle phase is clear and the bottom phase is slightly hazy, but essentially clear.

In Example 3, the phase volume separation is about 5% upper phase, 80% middle phase and 15% lower phase. The product separates into three layers fairly quickly (1 to 2 hours) but, it takes about one week for the top and middle layers to become clear while the bottom layer remains slightly hazy.

EXAMPLES 4, 5 & 6

| | EXAMPLE 4 | EXAMPLE 5 | EXAMPLE 6 |
|---|---|---|---|
| Ingredients | % | % | % |
| Sodium alpha | 30.0 | — | — |
| Olefin Sulfonate | | | |
| Ammonium Laureth | — | 30.0 | 30.0 |
| (3) Sulfate | | | |
| Cocamidopropyl | 30.0 | 30.0 | 30.0 |
| Betaine | | | |
| Mineral Oil (70 ssu) | 5.0 | 5.0 | — |
| Jojoba Oil | — | — | 5.0 |
| Dipropyleneglycol | 10.0 | 10.0 | 10.0 |
| Dibenzoate | | | |
| Propylene Glycol | 10.0 | 10.0 | 10.0 |
| Ammonium Xylene | 10.0 | 10.0 | 10.0 |
| Sulfonate | | | |
| Deionized Water | 5.0 | 5.0 | 5.0 |
| Fragrance, Color, | QS | QS | QS |
| Preservative | | | |
| | 100.0 | 100.0 | 100.0 |

In example 4, the upper phase is about 2.5%, the middle phase is about 90% and the bottom phase volume is about 7.5%. The upper and lower layers are clear, while the middle surfactant layer is slightly hazy. Separation occurs initially within several hours, however, it may take several days for the middle layer to clear. In example 5, the upper layer and lower layers are opaque, while the middle is slightly hazy. The upper layer and lower layer both have phase volumes of about 10% while the middle volume is about 80%. Example 6 has a hazy upper layer that represents about 15% of the liquid volume. There is a small (about 2.5% phase volume) slightly hazy bottom phase and the middle phase is slightly hazy.

EXAMPLES 7, 8 & 9

| | EXAMPLE 7 | EXAMPLE 8 | EXAMPLE 9 |
|---|---|---|---|
| Ingredients | % | % | % |
| Sodium alpha | — | — | 30.0 |
| Olefin Sulfonate | | | |
| Ammonium Laureth | 30.0 | 30.0 | — |
| (3) Sulfate | | | |
| Cocamidopropyl | 30.0 | — | 30.0 |
| Betaine | | | |
| Cocamidopropyl | — | 30.0 | — |
| Amine Oxide | | | |
| Jojoba Oil | 5.0 | 5.0 | 5.0 |
| Dipropyleneglycol | 10.0 | 10.0 | 10.0 |
| Dibenzoate | | | |
| Ethanol | 10.0 | 10.0 | 10.0 |
| Ammonium Xylene | 10.0 | 10.0 | 10.0 |
| Sulfonate | | | |
| Deionized Water | 5.0 | 5.0 | 5.0 |
| Fragrance, Color, | QS | QS | QS |
| Preservative | | | |
| | 100.0 | 100.0 | 100.0 |

In example 7, all three phases are clear with a phase volume ratio of about 7% to 88% to 5%; upper phase to middle phase to lower phase. Separation occurs initially within an hour, however, it may take several days for the middle layer to become completely clear. In example 8, the upper and middle layers are clear, but the lower layer is very slightly hazy. The phase volume separations are about the same as in example 7; i.e. 10% to 85% to 5%; for the upper, middle and bottom phases respectively. The phases begin separating within half an hour after shaking and clear within 24 hours. Example 9 has clear upper and lower layers with a very slightly hazy middle layer. The phase volume ratios are about 15% to 82.5% to 2.5%; top to middle to bottom.

EXAMPLES 10, 11, 12, 13

| | 10 | 11 | 12 | 13 |
|---|---|---|---|---|
| Ingredients | % | % | % | % |
| Ammonium Laureth (3) Sulfate | 30.0 | — | 30.0 | 30.0 |
| Sodium Laureth (3) Sulfate | — | 30.0 | — | — |
| Cocamidopropyl Betaine | 30.0 | 30.0 | 30.0 | — |
| Cocamidopropyl Hydroxy Sultaine | — | — | — | 30.0 |
| Jojoba Oil | — | — | 5.0 | 5.0 |
| GE 1202 Cyclomethicone | 10.0 | 10.0 | 3.0 | — |
| Dipropyleneglycol Dibenzoate | 10.0 | 10.0 | 10.0 | 10.0 |
| Ethanol | 10.0 | 10.0 | 10.0 | 10.0 |
| Sodium Chloride | — | — | 2.0 | — |
| Ammonium Xylene Sulfonate | 10.0 | 10.0 | 10.0 | 10.0 |
| Deionized water | — | — | — | 5.0 |
| Fragrance, Color, Preservative | QS | QS | QS | QS |
| | 100.0 | 100.0 | 100.0 | 100.0 |

Example10 has a slightly hazy 12.5% phase volume upper layer, an 80% phase volume middle layer that is clear and a 7.5% phase volume clear lower layer. Changing the surfactant counter ion from ammonium to sodium in Example 11, causes the upper phase volume to change to 35%, the middle to become 60% and the bottom layer to now represent only 5% of the total volume. Also, the upper and lower layers have become opaque while the middle one has become hazy. This is illustrative of the dramatic affects that simple formulation changes can have in these systems. Example 12 has three clear layers that have phase volumes of 10%, 82.5%, and 7.5% for the top, middle and bottom layers. Example 13 shows the same phase volume percentages as Example 12, but, the upper layer is slightly hazy. This formula foamed well in a hand washing test and would be suitable as a liquid hand soap product or body shampoo.

EXAMPLES 14, 15, 16 & 17

| | 14 | 15 | 16 | 17 |
|---|---|---|---|---|
| Ingredients | % | % | % | % |
| Ammonium Laureth (3) Sulfate | 30.0 | — | — | — |
| Ammonium Laureth (2) Sulfate | — | — | 30.0 | 30.0 |
| Sodium Laureth (3) Sulfate | — | 30.0 | — | — |
| Cocamidopropyl Betaine | 30.0 | 30.0 | 30.0 | — |
| Cocamidopropyl Hydroxy Sultaine | — | — | — | 30.0 |
| Jojoba Oil | 5.0 | 5.0 | 5.0 | 5.0 |
| GE 1202 Cyclomethicone | 5.0 | — | — | 1.0 |
| Dipropyleneglycol Dibenzoate | 10.0 | 10.0 | 10.0 | 12.0 |
| Ethanol | 10.0 | 10.0 | 10.0 | 10.0 |
| Ammonium Xylene Sulfonate | 10.0 | 10.0 | 10.0 | 10.0 |
| Deionized water | — | 5.0 | 5.0 | |
| Fragrance, Color, Preservative | QS | QS | QS | QS |
| | 100.0 | 100.0 | 100.0 | 100.0 |

Example 14 has three clear layers that represent respectively, 15%, 80% and 5% of the total volume. This product separates rapidly (within 30 minutes) and clears overnight. Example 15 also separates rapidly but has a slightly hazy upper layer with clear middle and lower layers. The phase volume percentages, however are 10%, 85% and 5% respectively. Example 16 shows phase volume separation percentages of 10%, 82.5% and 7.5% for the top, middle and bottom layers respectively. The top layer is slightly hazy, while the other two are clear. The final Example, # 17, has three clear layers in the ratio of 10%, 85% and 5% for the top, middle and bottom layers respectively.

It is to be understood by those skilled in the art that the foregoing descriptions and examples are illustrative of practicing the present invention, but are in no way limiting. Variations of the details presented herein may be made without departing from the spirit and scope of the present invention as defined by the following claims.

What is claimed:

1. A multi-phase surfactant composition wherein said composition comprises discrete phases upon settling, said composition comprising a first or lower high density emollient/conditioning layer comprising at least one high density aromatic ester emollient or conditioning agent having a specific gravity of greater than 1.00 and at least one additional layer comprising a surfactant solution having a specific gravity of less than the specific gravity of said high density layer.

2. The composition according to claim 1 comprising three (3) distinct phases, a first layer comprising at least one high density aromatic ester emollient or conditioning agent having a specific gravity of greater than 1.00; a second layer comprising a surfactant solution comprising water and a foaming surfactant having a specific gravity ranging from about 0.90 to about 1.05 and being less than the specific gravity of said first layer; and a third or upper layer comprising a low density oily material having emollient or conditioning characteristics and a specific gravity ranging from about 0.7 to about 0.95, said third layer having a specific gravity less than said first layer and said second layer.

3. The composition according to claim 2 wherein said composition includes a fourth phase comprising at least one exfoliating agent.

4. The composition according to claim 2 wherein said phases exist as three discrete liquid layers lying one on top of another.

5. The composition according to claim 2 wherein said first layer has a specific gravity of greater than about 1.0, said second layer has a specific gravity ranging from about 0.95 to about 1.00 and said third layer has a specific gravity ranging from about 0.8 to about 0.95, the specific gravity of said third layer being less than the specific gravity of said second layer and the specific gravity of said second layer being less than the specific gravity of said first layer.

6. The composition according to claim 3 wherein said fourth layer has a specific gravity which is greater than said first layer.

7. The composition according to claim 3 wherein said fourth phase is a solid phase comprising a solid particulate material selected from the group consisting of ground plant materials, finely divided polyethylene, hydrogenated jojoba oil spheres, diatomaceous earth, sand, pumice and mixtures, thereof.

8. The composition according to claim 1 wherein said high density aromatic esters are selected from the group consisting of propylene glycol benzoate, dipropylene glycol dibenzoate, dipropylene glycol dibenzoate, dipropylene glycol benzoate, octyl methoxycinnamate, menthyl anthranilate, octyl salicylate, octyl cinnamate, and octocrylene.

9. The composition according to claim 3 wherein said high density aromatic esters are selected from the group consisting of propylene glycol benzoate, dipropylene glycol benzoate, dipropylene glycol dibenzoate, propylene glycol dibenzoate, octyl methoxycinnamate, menthyl anthranilate, octyl salicylate, octyl cinnamate, and octocrylene.

10. The composition according to claim 2 wherein said third layer comprises at least one low density emollient or conditioning agent selected from the group consisting of mineral oil, squalane, squalene, difatty esters such as Jojoba oil, oleyl oleate, oleyl erucate, polydimethylsiloxane and cyclomethicone.

11. The composition according to claim 1, wherein said surfactant solution comprises an anionic surfactant selected from the group consisting of alkyl sulfates, alkylether sulfates, alkyl benzene sulfonates, alpha olefin sulfonates, N-alkyl sarcosinates, alkyl sulfosuccinates, alkyl phosphates, alkylether phosphates and alkyl and alkylether carboxylic acid salts.

12. The composition according to claim 2, wherein said surfactant solution comprises an anionic surfactant selected from the group consisting of alkyl sulfates, alkylether sulfates, alkyl benzene sulfonates, alpha olefin sulfonates, N-alkyl sarcosinates, alkyl sulfosuccinates, alkyl phosphates, alkylether phosphates and alkyl and alkylether carboxylic acid salts.

13. The composition according to claim 1 wherein said surfactant solution comprises a surfactant selected from the group consisting of anionic, cationic, nonionic, amphoteric and zwitterionic surfactants.

14. The composition according to claim 2 wherein said surfactant solution comporises a surfactant selected from the group consisting of cationic, nonionic, amphoteric and zwitterionic surfactants.

15. The composition according to claim 3 wherein said surfactant solution comporises a surfactant selected from the group consisting of cationic, nonionic, amphoteric and zwitterionic surfactants.

16. The composition according to claim 13 wherein said surfactant may be used to modify the physical properties of the surfactant solution phase to change its feel, viscosity, clarity, foaming, foam stability, cloud point, skin and hair conditioning and/or its detergency.

17. The composition according to claim 14 wherein said surfactant may be used to modify the physical properties of the surfactant solution phase to change its feel, viscosity, clarity, foaming, foam stability, cloud point, skin and hair conditioning and/or its detergency.

18. A composition according to claim 1 wherein said surfactant solution may comprise one or more separation enhancer.

19. The composition according to claim 18 wherein said separation enhancer is selected from the group consisting of ethanol, isopropanol, propanol, propyleneglycol, methyl propane diol, butylene glycols, dipropylene glycol, ethoxy diglycol, hexylene glycol, xylene sulfonate salts, naphthalene sulfonate salts, acetamido and lactamido propyl trimonium chlorides, sorbitol, glucose, sucrose, fructose, glycerin, ethoxylated glycerin, ethoxylated glycols or a salt formed from a cation and an anion wherein said cation is selected from the cation of sodium, ammonium, potassium, calcium and magnesium and said anion is selected from the anion of chloride, sulfate, nitrate, phosphate, carbonate, acetate, lactate and ethylenediaminetetraacetate.

20. A composition according to claim 2 wherein said surfactant solution may comprise one or more separation enhancer.

21. The composition according to claim 20 wherein said separation enhancer is selected from the group consisting of ethanol, isopropanol, propanol, propyleneglycol, methyl propane diol, butylene glycols, dipropylene glycol, ethoxy diglycol, hexylene glycol, xylene sulfonate salts, naphthalene sulfonate salts, acetamido and lactamido propyl trimonium chlorides, sorbitol, glucose, sucrose, fructose, glycerin, ethoxylated glycerin, ethoxylated glycols or a salt formed from a cation and an anion wherein said cation is selected from the cation of sodium, ammonium, potassium, calcium and magnesium and said anion is selected from the anion of chloride, sulfate, nitrate, phosphate, carbonate, acetate, lactate and ethylenediaminetetraacetate.

22. The composition according to claim 1 wherein said high density aromatic ester is selected from the group consisting of benzoic acid esters of diols, triols and tetraols.

23. The composition according to claim 2 wherein said high density aromatic ester is selected from the group consisting of benzoic acid esters of diols, triols and tetraols.

24. The composition according to claim 1 wherein said aromatic ester is selected from the group consisting of menthyl anthranilate, octocrylene, octyl methoxycinnamate, octyl salicylate and mixtures, thereof.

25. The composition according to claim 2 wherein said aromatic ester is selected from the group consisting of menthyl anthranilate, octocrylene, octyl methoxycinnamate, octyl salicylate and mixtures, thereof.

26. The composition according to claim 2 wherein said low density oily material is selected from the group consisting of mineral oils, alphatic hydrocarbons, branched aliphatic hydrocarbons, squalane, squalene, cyclomethicone, jojoba oil andr monoesters of fatty acids and fatty alcohols containing a total of from 36 to 42 total carbons.

27. The composition according to claim 1 further comprising a third phase comprising a solid exfoliating agent.

28. The composition according to claim 1 formulated as a hair shampoo, a body cleanser or a hand cleanser.

29. The composition according to claim 2 formulated as a hair shampoo, a body cleaner or a hand cleanser.
